(19) United States
(12) Patent Application Publication
(10) Pub. No.: US 2019/0038887 A1
(43) Pub. Date: Feb. 7, 2019

(54) Medical drain unit having luer-lock connection structure

(71) Applicant: Hyun Woo LEE, Hanam-si (KR)

(72) Inventor: Hyun Woo LEE, Hanam-si (KR)

(21) Appl. No.: 16/072,523

(22) PCT Filed: Nov. 11, 2016
(86) PCT No.: PCT/KR2016/012964

§ 371 Date: Jul. 25, 2018

(30) Foreign Application Priority Data
Oct. 5, 2016 (KR) 10-2016-0128539
Nov. 4, 2016 (KR) 10-2016-0146805

(51) Int. Cl. A61M 39/10; A61J 1/10; A61J 1/20; A61M 5/40

(52) U.S. Cl. CPC A61M 39/1011; A61J 1/10; A61M 39/24; A61M 5/40; A61J 1/20

(57) ABSTRACT

The present invention relates to a medical drain unit comprising: a drain port installed in an infusion solution container; and a drip chamber detachably coupled to the drain port and discharging an infusion solution stored in the infusion solution container. More specifically, the present invention relates to a medical drain unit having a luer-lock connection structure, in which a rubber stopper having a cut incision and a first coupling pipe exposing the incision are installed in the drain port; and a second coupling pipe coupled to the first coupling pipe and a luer-lock connector including an insertion tube which is inserted into the incision to discharge the infusion solution are installed in the drip chamber, whereby a spike that is forcedly inserted into the rubber stopper is not required.

### TECHNICAL FIELD

The present invention relates to a medical drain unit having a luer-lock connection structure that includes a drain port disposed on an infusion solution container and a drip chamber detachably coupled to the drain port to discharge an infusion solution stored in the infusion solution container therefrom, and more specifically, to a medical drain unit having a luer-lock connection structure wherein a drain port includes a rubber stopper having an incision line formed thereon and a first coupling tube adapted to expose the incision line to the outside, and a drip chamber includes a luer-lock connector having a second coupling tube coupled to the first coupling tube pipe, an insertion tube inserted into the incision line to discharge an infusion solution, so that no spike forcedly inserted into the rubber stopper is required.

### BACKGROUND ART

FIG. 1 shows a general structure of a conventional infusion set used for medical purposes, and the infusion set includes an infusion solution container 100 storing an infusion solution therein, a drain port 110 disposed on the infusion solution container, a drip chamber 120 discharging the infusion solution from the infusion solution container 100, an infusion tube 140 connected to the underside of the drip chamber 120, and a needle 150 and a needle connector 160 disposed on the end of the infusion tube 140. The drip chamber 120 of the infusion set, which is also called ‘dropping tube’, serves to discharge the infusion solution stored in the infusion solution container 100 through the insertion of a spike into the drain port 110,. The drip chamber 120 is made of a soft transparent material to check a speed of infusion solution discharged by naked eyes, and both sides of the drip chamber 120 can be pressedly pumped to accelerate the discharging speed of the infusion solution. Further, the drain port 110 has a rubber stopper adapted to forcedly insert the sharp spike of the drip chamber 120 thereinto, and accordingly, the infusion solution discharged through the spike is temporarily stored in the drip chamber 120 and is then discharged to the infusion tube 140.

According to the conventional infusion set, by the way, when the spike is piercedly inserted into the rubber stopper of the drain port 110, tiny rubber pieces are generated from the rubber stopper, and if the rubber pieces are introduced into a patient's body, together with the infusion solution, serious side effects may occur on the patient's body.

To solve the above-mentioned problems, an infusion set is disclosed in Korean Patent Application Laid-open No.10-2012-0035260 (Dated on Apr. 16, 2012), wherein a filter member is disposed on a Y-shaped branch tube of an infusion tube so as to filter foreign matters like rubber pieces generated when a spike is inserted into a rubber stopper, and further, a drain unit is disclosed in Korean Patent Application Laid-open No. 10-2015-168350 (Dated on Nov. 30, 2015), wherein a filter member is disposed in a drip chamber.

In case of the conventional drip chamber using the spike, moreover, if unexpected tension is applied to the infusion tube in a process where the infusion solution is discharged, the spike may escape from the drain port, or safety accidents may happen so that a nurse or patient is injured by the spike.

Also, other conventional drip chambers have been suggested in Korean Utility Model Registration No. 20-0458702 (Dated on Feb. 9, 2012) and Korean Utility Model Registration No. 20-0470517 (Dated on Dec. 16, 2013), and a medical connector having a lure-lock structure has been proposed in Korean Patent No. 10-1329056 (Dated on Nov. 7, 2013).

### PRIOR ART DOCUMENTS

(Patent document 1) Korean Utility Model Registration No. 20-0458702 (Dated on Feb. 9, 2012)

(Patent document 2) Korean Utility Model Registration No. 20-0470517 (Dated on Dec. 16, 2013)

(Patent document 3) Korean Patent No. 10-1329056 (Dated on Nov. 7, 2013)

### DISCLOSURE

### Technical Problem

Accordingly, the present invention has been made in view of the above-mentioned problems occurring in the prior art, and it is an object of the present invention to provide a medical drain unit having a luer-lock connection structure that is capable of preventing rubber pieces from being generated by means of a sharp spike in a process where a drain port and a drip chamber in an infusion set are connected to each other and capable of avoiding dangers of safety accidents caused by the sharp spike.

It is another object of the present invention to provide a medical drain unit having a luer-lock connection structure that is capable of preventing a connected state between a drain port and a drip chamber from being easily released even though unexpected tension is applied to an infusion tube in a process where an infusion solution is discharged from an infusion solution container.

### Technical Solution

To accomplish the above-mentioned objects, according to the present invention, there is provided a medical drain unit having a luer-lock connection structure, includes: a drain port disposed in an infusion solution container; and a drip chamber detachably coupled to the drain port to discharge an infusion solution stored in the infusion solution container, wherein the drain port includes a rubber stopper having an incision line formed thereon and a first coupling tube adapted to expose the incision line to the outside, and the drip chamber includes a luer-lock connector having a second coupling tube coupled to the first coupling tube and an insertion tube inserted into the incision line to discharge the infusion solution from the infusion solution container, the drip chamber having no spike forcedly inserted into the rubber stopper.

According to the present invention, desirably, the drain port includes a drain tube connected to the infusion solution container (A1) to discharge the infusion solution from the infusion solution container (A1), the rubber stopper disposed on an inner lower end periphery of the drain tube and having a center hole formed thereon in upward and downward direction and the incision line formed on a bottom portion shielding a bottom end periphery of the center hole, and a rubber stopper cap member coupled to the lower end periphery of the drain tube to surround the rubber stopper and having a first coupling tube protruding from the lower end periphery thereof to expose the incision line to the outside;

According to the present invention, desirably, the luer-lock connector includes the second coupling tube coupled to the outer peripheral surface of the first coupling tube, a sleeve enlarged in diameter from the lower end periphery of the second coupling tube in such a manner as to be extended downward therefrom, and the insertion tube adapted to pass through centers of the second coupling tube and the sleeve in upward and downward directions in such a manner as to allow an upper end periphery thereof to pass through the incision line.

According to the present invention, desirably, the drip chamber with a cylindrical shape includes an internal space in which the infusion solution dropped from the insertion tube is temporarily stored, an upper end periphery thereof fixedly coupled to the outer peripheral surface of the sleeve, and an infusion solution discharge hole formed on a bottom end thereof in such a manner as to be connected to an infusion tube (A2).

According to the present invention, desirably, the drip chamber includes a floating member disposed in an interior thereof in such a manner as to be ascended and descended according to the level of the infusion solution and, if the infusion solution is all discharged, to shield a top of the infusion solution discharge hole.

### Advantageous Effects

According to the present invention, the medical drain unit having the luer-lock connection structure does not use any spike at the time when the drain port and the drip chamber are connected to inject the infusion solution into a patient's body, so that there is no possibility that rubber pieces are generated in the spike insertion process, a difficulty in pushing the rubber stopper or inserting the spike into the rubber stopper can be removed, and the nurse or patient cannot be injured by the sharp spike to prevent the generation of safety accidents.

In addition, the medical drain unit having the luer-lock connection structure according to the present invention is configured to allow the first coupling tube of the drain port and the second coupling tube of the lure-lock connector to be rigidly coupled to each other, so that even though unexpected tension is applied to the infusion tube connected to the underside of the drip chamber in the process where the infusion solution is discharged from the infusion solution container, there is no possibility that the connected state between the drain port and the drip chamber is released.

### DESCRIPTION OF DRAWINGS

FIG. 1 is a perspective view showing a structure of a conventional infusion set.

FIG. 2 is a sectional view showing a structure of a medical drain unit according to the present invention.

FIGS. 3a to 6b are perspective and sectional views showing various examples of a drain port of the medical drain unit according to the present invention.

FIGS. 7a and 7b are perspective and sectional views showing the coupled state between a lure-lock connector and a drip chamber of the medical drain unit according to the present invention.

FIG. 8 is a sectional view showing a state where the drain port and the lure-lock connector are connected to each other and a floating member is disposed in the drip chamber in the medical drain unit according to the present invention.

FIG. 9 is a sectional view showing another coupled state between the lure-lock connector and the drip chamber of the medical drain unit according to the present invention.

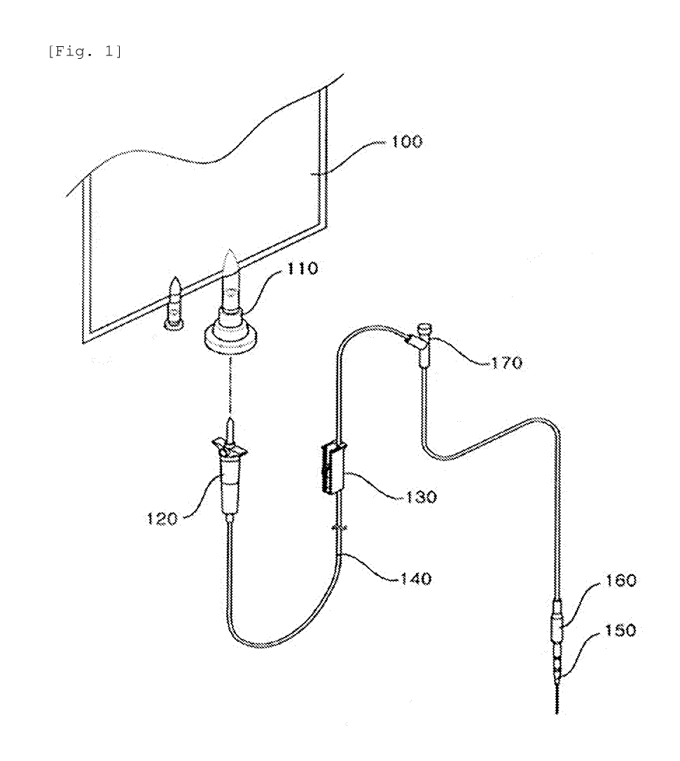

<Explanation on reference numerals in the drawings>

| | |
|---|---|
| 10, 10a, 10b, 10c, 10d: drain port | |
| 11: drain tube | 111: enlarged portion |
| 12a, 12b: rubber stopper | 121: center hole |
| 122: incision line | |
| 13a, 13b: rubber stopper cap member | |
| 131: first coupling tube | |
| 14a, 14b: rubber stopper support member | |
| 141: upper round wall | 142: lower round wall |
| 143: rubber stopper support projection | |
| 20, 20a, 20b: lure-lock connector | |
| 21: second coupling tube | |
| 22: sleeve | 23: insertion tube |
| 24: ventilation hole | 25: air filter |
| 26: connection port | 30: drip chamber |
| 31: infusion solution discharge hole | |
| 40: floating member | |
| 41: ascending/descending cup | |
| 42: infusion solution passing hole | |
| 43: shut-off valve | |

### BEST MODE FOR INVENTION

Hereinafter, the present invention will now be described in detail with reference to the attached drawings. Before the present invention is disclosed and described, it is to be understood that the disclosed embodiments are merely exemplary of the invention, which can be embodied in various forms. If it is determined that the detailed explanation on the well known technology related to the present invention makes the scope of the present invention not clear, the explanation will be avoided for the brevity of the description.

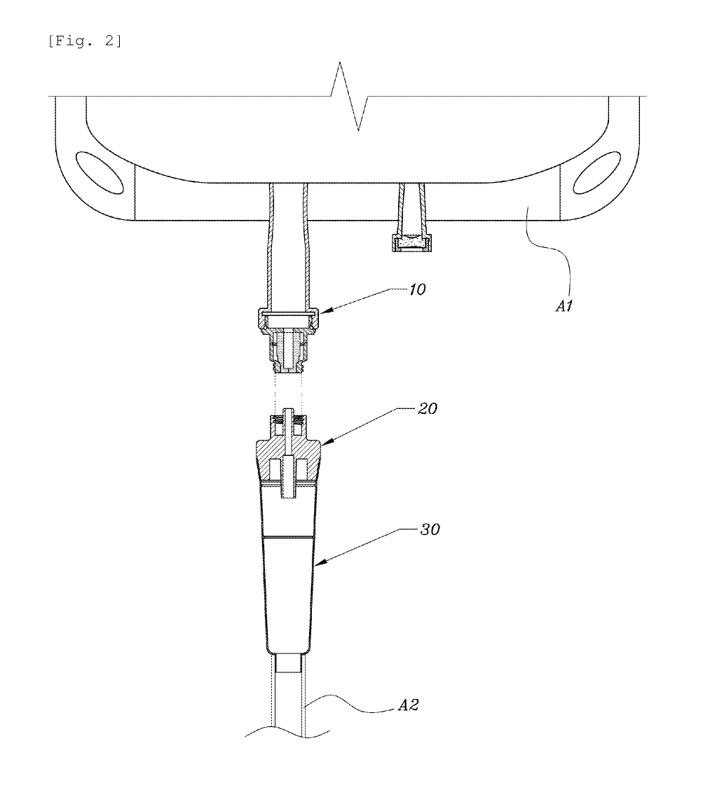

For the convenience of the description, around FIG. 2, the side at which an infusion solution container A1 is located is called ‘upper side’ or ‘upward side’, and the side at which an infusion solution tube A2 is located is called ‘lower side’ or ‘downward side’. Even though the corresponding parts in the embodiments of the present invention have somewhat different structures, the parts having the same functions as each other are indicated by corresponding reference numerals.

As shown in FIG. 2, a medical drain unit according to the present invention includes a drain port 10 disposed on an infusion solution container A1, a lure-lock connector 20 detachably coupled to a lower end periphery of the drain port 10, and a drip chamber 30 fixedly coupled to a lower end periphery of the lure-lock connector 20. The infusion solution container A1 is desirably used with a plastic bag sealedly made of a synthetic resin film made of a non-PVC material, but of course, it is not limited necessarily thereto. A general infusion tube A2 is connected to a lower end periphery of the drip chamber 30.

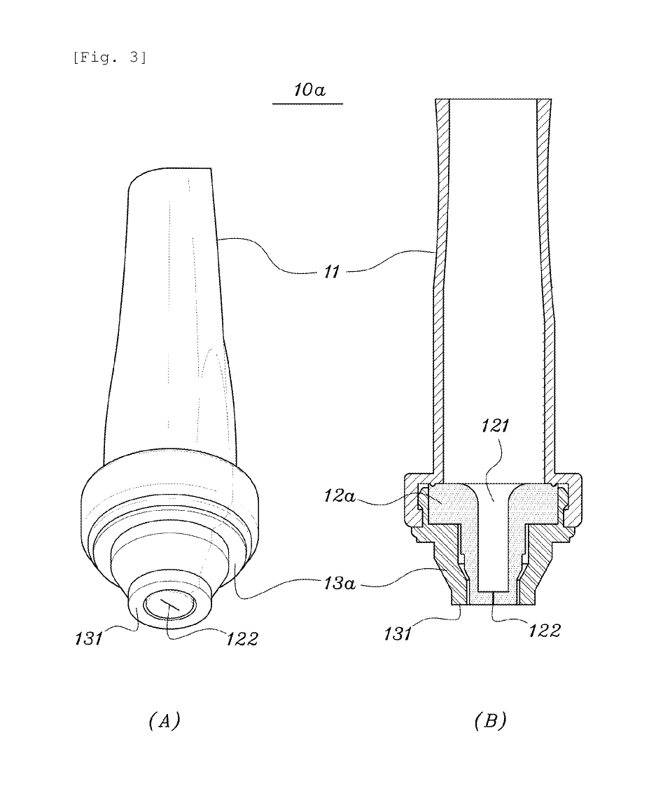

FIGS. 3a and 3b show a drain port 10a as an example of the drain port 10 according to the present invention, wherein the drain port 10a includes a drain tube 11, a rubber stopper 12a, and a rubber stopper cap member 13a. The drain tube 11 is connected to the infusion solution container A1. As shown in FIG. 2, the drain tube 11 is directly sealedly connected to the infusion solution container A1 or inserted into a connection tube (not shown) sealed in the infusion solution container A1.

If the drain tube 11 is directly sealedly connected to the infusion solution container A1, it may have a sectional shape of a flat diamond, or it has a shape of an oval having both side ear portions protruding therefrom, as shown in FIGS. 3a and 3b, so as to prevent an infusion solution from leaking from the sealed portion between the drain tube 11 and the infusion solution container A1. If the drain tube 11 is inserted into the connection tube (not shown) sealed in the infusion solution container A1, on the other hand, it has a sectional shape of a circle.

The rubber stopper 12a is disposed on an inner lower end periphery of the drain tube 11 and has a center hole 121 formed thereon in upward and downward direction. A top end periphery of the center hole 121 is connected to the drain tube 11, and a bottom end periphery thereof is shielded by a bottom portion of the rubber stopper 12a. An incision line 122 is formed on the bottom portion of the rubber stopper 12a shielding the bottom end periphery of the center hole 121.

Like a general medical rubber stopper, the rubber stopper 12a is made of rubber or elastomer which gives no harm to the body of a human and has excellent elasticity, and the incision line 122 is desirably formed to a shape of a cross (+) or straight line (−). If an appropriate external force is applied to the incision line 122, the incision line 122 may be open, but if the external force disappears, it is returned to its original state by means of the elasticity of the material itself.

The rubber stopper cap member 13a is coupled to the lower end periphery of the drain tube 11 to surround the rubber stopper 12a, thereby serving to prevent the rubber stopper 12a from escaping from the drain tube 11. As shown in FIGS. 3a and 3b, a first coupling tube 131 protrudes from the lower end periphery of the rubber stopper cap member 13a to expose the incision line 122 to the outside.

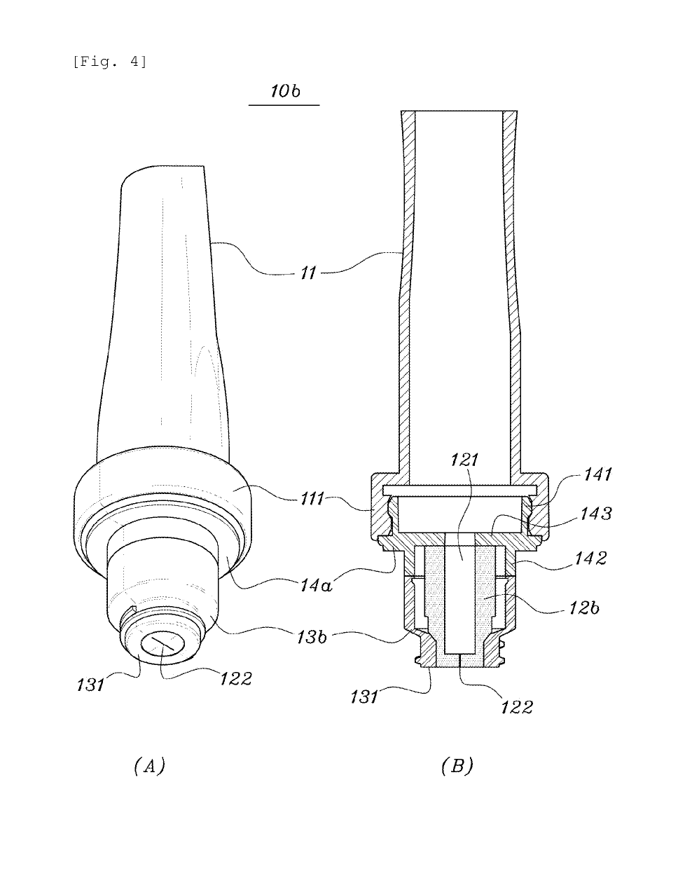

FIGS. 4a and 4b show a drain port 10b as another example of the drain port 10 according to the present invention. In this case, the drain tube 11 has an enlarged portion 111 formed enlarged in diameter on the lower end periphery thereof, and a rubber stopper support member 14a is additionally disposed between the drain tube 11 and a rubber stopper cap member 13b. The rubber stopper support member 14a has an upper round wall 141 inserted into the enlarged portion 111, a lower round wall 142 coupled to the rubber stopper cap member 13b, and a rubber stopper support projection 143 formed on the inner peripheral surface between the upper round wall 141 and the lower round wall 142 to support the upper end periphery of a rubber stopper 12b.

In the state where the drain tube 11, the rubber stopper cap member 13b, and the rubber stopper support member 14a are injection-molded independently of each other, the rubber stopper 12b is mounted in the interior of the drain tube 11 and is coupled integrally with the drain tube 11, the rubber stopper cap member 13b, and the rubber stopper support member 14a. The rubber stopper 12b and the rubber stopper cap member 13b in FIGS. 4a and 4b are somewhat differently configured from the rubber stopper 12a and the rubber stopper cap member 13a in FIGS. 3a and 3b, but their functions are the same as each other. Further, a protection cap (not shown) is mounted on the first coupling tube 131 to prevent the incision line 122 from being contaminated.

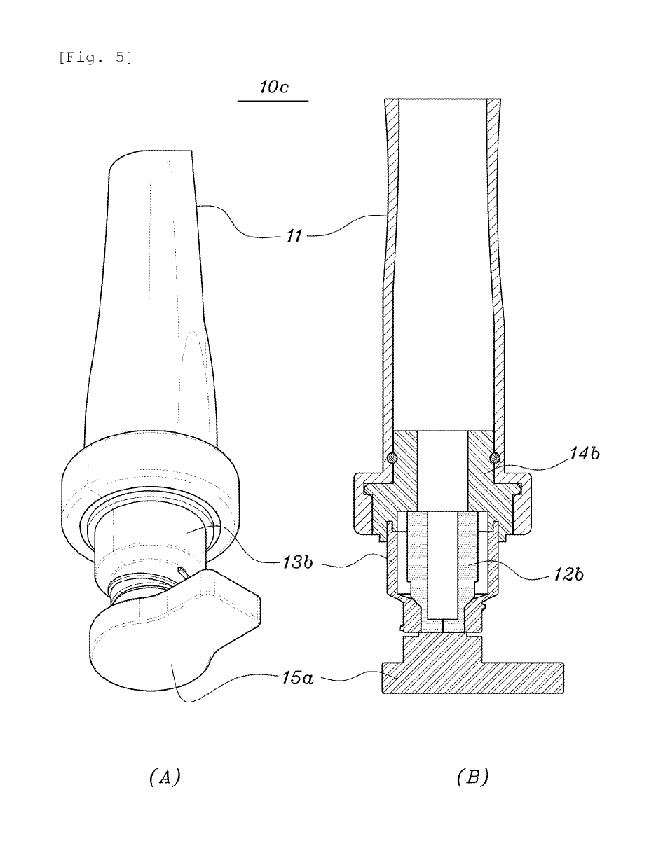

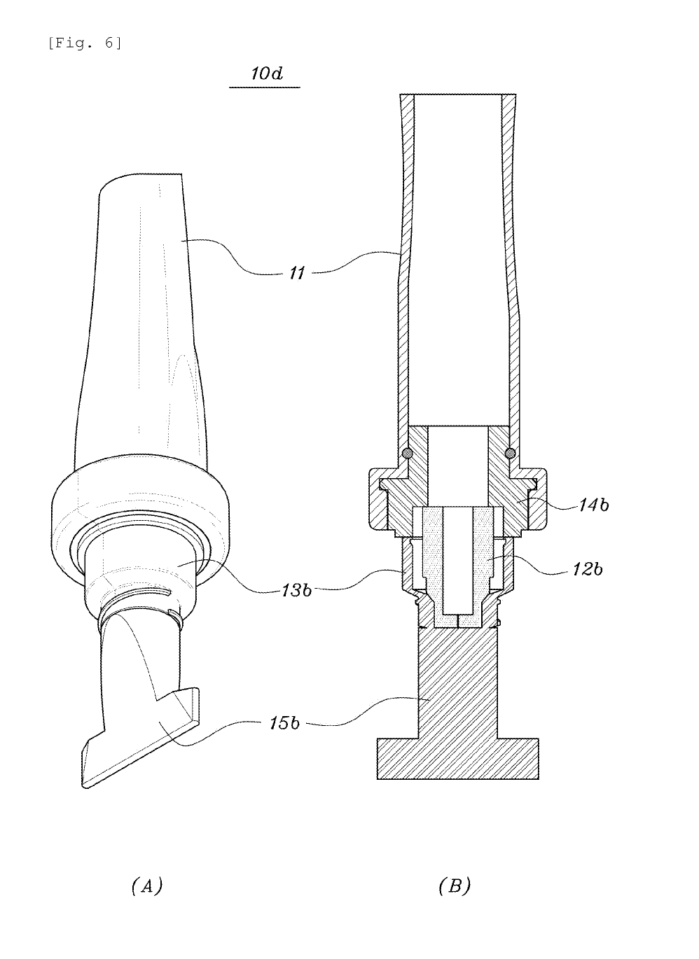

FIGS. 5a to 6b show drain ports 10c and 10d as other examples of the drain port 10 according to the present invention, and in this case, a cut-off cap 15a or 15b is mounted on the underside of the first coupling tube 131 to protect the incision line 122 from the outside. The cut-off caps 15a and 15b are simply cut off by means of one touch if necessary, thereby exposing the incision line 122 to the outside. The cut-off caps 15a and 15b are somewhat differently configured from each other, but their functions are the same as each other. The rubber stopper support members 14b in FIGS. 5a to 6b are somewhat differently configured from the rubber stopper support member 14a in FIGS. 4a and 4b, but their functions are the same as each other.

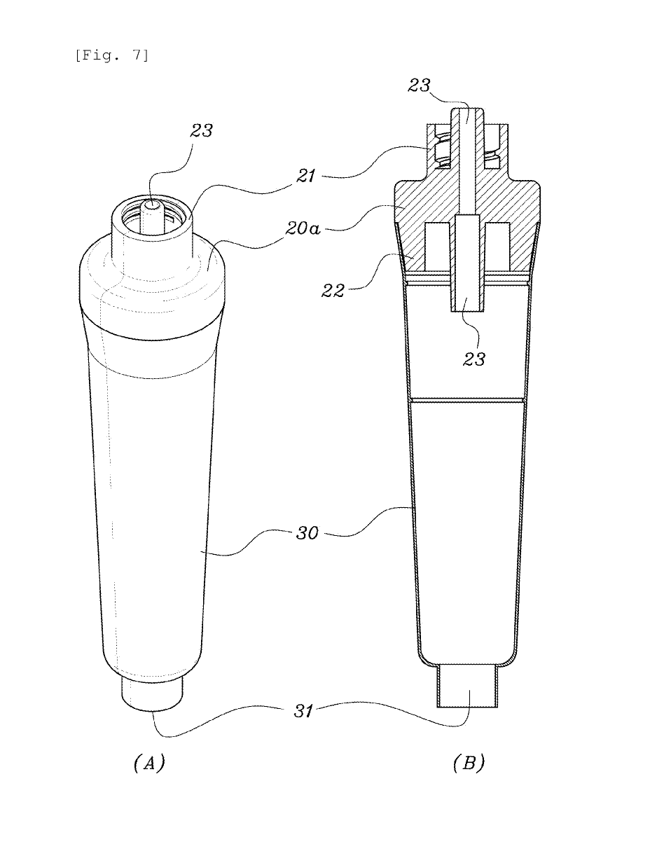

Next, the lure-lock connector 20 is detachably coupled to the lower end periphery of the drain port 10 and serves to discharge the infusion solution stored in the infusion solution container A1. According to the present invention, as shown in FIGS. 7a and 7b, the lure-lock connector 20 includes a second coupling tube 21 coupled to the first coupling tube 131 of the rubber stopper cap member 13a or 13b, a sleeve 22 enlarged in diameter from the lower end periphery of the second coupling tube 21 in such a manner as to be extended downward therefrom, and an insertion tube 23 adapted to pass through centers of the second coupling tube 21 and the sleeve 22 in upward and downward directions.

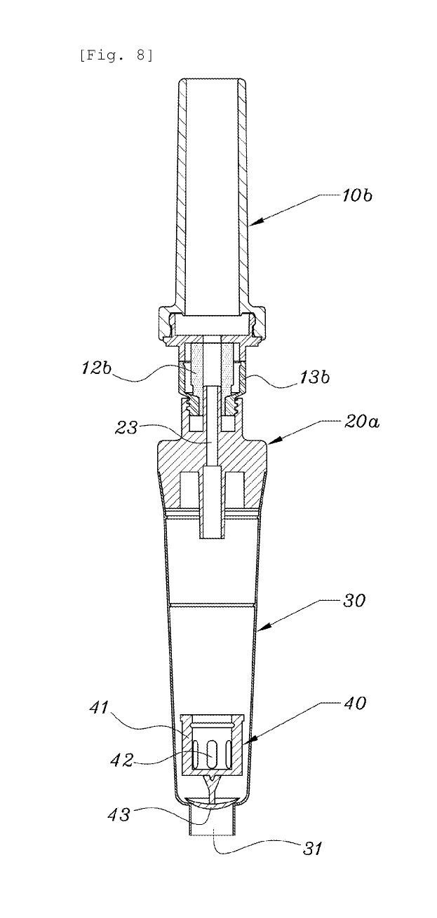

The first coupling tube 131 and the second coupling tube are tightly coupled to each other by means of forced insertion, and otherwise, they are screw-coupled to each other by means of female and male screws formed correspondingly thereon. If the second coupling tube 21 is coupled to the first coupling tube 131, as shown in FIG. 8, an upper end periphery of the insertion tube 23 is inserted into the first coupling tube 131, passes through the incision line 122 of the rubber stopper 12a or 12b, and is finally connected to the center hole 121.

At this time, the incision line 122 is in advance formed on the rubber stopper 12a or 12b, so that the insertion tube 23 can be easily inserted into the rubber stopper 12a or 12b. A given gap is formed between the inner peripheral wall of the rubber stopper cap member 13a or 13b and the rubber stopper 12b, so that it accommodates a portion of the rubber stopper 12b whose outer diameter becomes enlarged by means of the insertion of the insertion tube 23.

The drip chamber 30 is of a shape of a cylinder and is made of a transparent or semi-transparent material. The drip chamber 30 has an internal space in which the infusion solution dropped from the insertion tube 23 is temporarily stored. An upper end periphery of the drip chamber 30 is coupled to the sleeve 22, and an infusion solution discharge hole 31 is formed on a bottom end of the drip chamber 30 in such a manner as to be connected to the infusion tube A2. The drip chamber 30 is made of a soft polypropylene or polyethylene resin having a restoring force to its original shape, so that if necessary, the infusion solution stored in the drip chamber 30 can be pumped.

On the other hand, as shown in FIG. 8, a floating member 40 is disposed in the drip chamber 30 in such a manner as to be ascended and descended according to the level of the infusion solution and, if the infusion solution is all discharged, to block a top of the infusion solution discharge hole 31 to prevent introduction of air. The floating member 40 includes an ascending/descending cup 41 facing the inner peripheral wall of the drip chamber 30 in such a manner as to be spaced apart therefrom, infusion solution passing holes 42 pierced on the ascending/descending cup 41 in a circumferential direction of the ascending/descending cup 41, and a shut-off valve 43 protruding downward from the underside of the ascending/descending cup 41.

The ascending/descending cup 41 has a buoyant force with respect to the infusion solution and is desirably made of a material giving no harm to the body of a human, for example, a polypropylene resin. The specific gravity of polypropylene is lower than that of the infusion solution and thus has the buoyant force with respect to the infusion solution.

The infusion solution passing holes 42 serve as passages along which the infusion solution introduced into the insertion tube 23 flows to the lower end portion of the drip chamber 30. If the level of the infusion solution is raised in the drip chamber 30, the floating member 40 is ascended to allow the infusion solution discharge hole 31 to be open, and contrarily, if the level of the infusion solution is lowered in the drip chamber 30, the floating member 40 is descended to allow the infusion solution discharge hole 31 to be closed.

The shut-off valve 43 has a shape of a dish in such a manner as to become reduced in thickness as it goes toward the edge periphery thereof from the center thereof. Further, the shut-off valve 43 is desirably made of soft elastomer, silicone, or rubber.

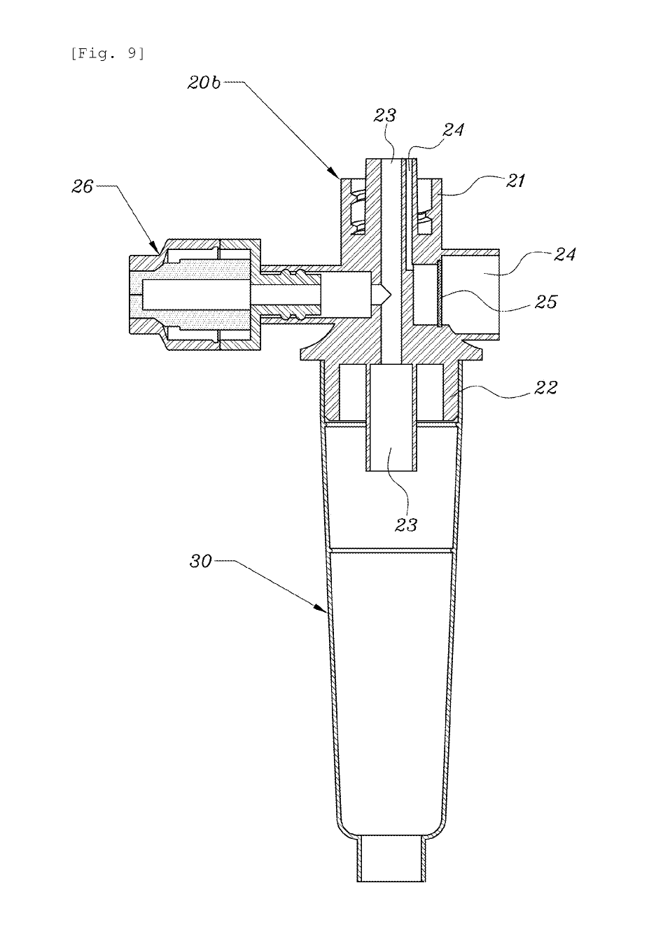

FIG. 9 is a sectional view showing another example of the lure-lock connector 20. In this case, a lure-lock connector 20b is configured to have a ventilation hole 24 connected to the top of the insertion tube 23 from one side between the second coupling tube 21 and the sleeve 22 and an air filter 25 disposed in the ventilation hole 24. The ventilation hole 24 serves to supply air to the infusion solution container A1 to easily discharge the infusion solution from the infusion solution container A1 even though the infusion solution container A1 is a hard container having no shrinkage.

Further, the lure-lock connector 20b is configured to have a connection port 26 disposed between the second coupling tube 21 and the sleeve 22. The connection port 26 serves to connect a connector adapted to mixedly introduce another infusion solution or drug into the insertion tube 23 thereto. As shown in FIG. 9, the structure of the connection port 26 is similar to that of the drain port 10b of FIGS. 5a and 5b, and a detailed explanation on the connection port 26 will be avoided.

1. A medical drain unit having a luer-lock connection structure, comprising:
a drain port having a drain tube connected to an infusion solution container to discharge an infusion solution from the infusion solution container, a rubber stopper disposed on an inner lower end periphery of the drain tube and having a center hole formed thereon in upward and downward direction and an incision line formed on a bottom portion shielding a bottom end periphery of the center hole, and a rubber stopper cap member coupled to the lower end periphery of the drain tube to surround the rubber stopper and having a first coupling tube protruding from the lower end periphery thereof to expose the incision line to the outside;
a luer-lock connector having a second coupling tube coupled to the outer peripheral surface of the first coupling tube, a sleeve enlarged in diameter from the lower end periphery of the second coupling tube in such a manner as to be extended downward therefrom, and an insertion tube adapted to pass through centers of the second coupling tube and the sleeve in upward and downward directions in such a manner as to allow an upper end periphery thereof to pass through the incision line; and
a drip chamber with a cylindrical shape having an internal space in which the infusion solution dropped from the insertion tube is temporarily stored, an upper end periphery thereof fixedly coupled to the outer peripheral surface of the sleeve, and an infusion solution discharge hole formed on a bottom end thereof in such a manner as to be connected to an infusion tube.

2. The medical drain unit according to claim 1, wherein the drain tube is directly sealedly connected to the infusion solution container, and the drain tube has a sectional shape of a flat diamond or a shape of an oval having both side ear portions protruding therefrom.

3. The medical drain unit according to claim 1, wherein a rubber stopper support member is additionally disposed between the drain tube and the rubber stopper cap member, the drain tube has an enlarged portion formed enlarged in diameter on the lower end periphery thereof, and the rubber stopper support member comprises an upper round wall inserted into the enlarged portion, a lower round wall coupled to the rubber stopper cap member, and a rubber stopper support projection adapted to support the upper end periphery of the rubber stopper thereagainst.

4. The medical drain unit according to claim 1, wherein the first coupling tube has a cut-off cap mounted on the underside thereof to protect the incision line from the outside in such a manner as to be cut off if necessary to expose the incision line to the outside.

5. The medical drain unit according to claim 1, wherein the first coupling tube has a male screw formed on the outer peripheral surface thereof and the second coupling tube has a female screw formed on the inner peripheral surface thereof in such a manner as to be coupled correspondingly to the male screw of the first coupling tube.

6. The medical drain unit according to claim 1, wherein the drip chamber has a floating member disposed in an interior thereof in such a manner as to be ascended and descended according to the level of the infusion solution and, if the infusion solution is all discharged, to shield a top of the infusion solution discharge hole to prevent introduction of air.

7. The medical drain unit according to claim 6, wherein the floating member comprises: an ascending/descending cup facing the inner peripheral wall of the drip chamber in such a manner as to be spaced apart therefrom; infusion solution passing holes pierced on the ascending/descending cup in a circumferential direction of the ascending/descending cup; and a shut-off valve protruding downward from the underside of the ascending/descending cup in such a manner as to come into close contact with an entrance of the infusion solution discharge hole.

8. The medical drain unit according to claim 1, wherein the lure-lock connector comprises a ventilation hole connected to the top of the second coupling tube from the side between the second coupling tube and the sleeve and an air filter disposed in the ventilation hole.

9. The medical drain unit according to claim 1, wherein the lure-lock connector comprises a connection port protruding from one side between the second coupling tube and the sleeve in such a manner as to connect a connector adapted to mixedly introduce another infusion solution or drug into the drip chamber to one side.

10. A medical drain unit having a luer-lock connection structure, comprising:
a drain port disposed on an infusion solution container; and
a drip chamber detachably coupled to the drain port to discharge an infusion solution stored in the infusion solution container,
wherein the drain port comprises a rubber stopper having an incision line formed thereon and a first coupling tube adapted to expose the incision line to the outside, and the drip chamber comprises a luer-lock connector having a second coupling tube coupled to the first coupling tube and an insertion tube inserted into the incision line to discharge the infusion solution from the infusion solution container, the drip chambering having no spike forcedly inserted into the rubber stopper.
